(19) United States
(12) Patent Application Publication
(10) Pub. No.: US 2009/0299399 A1
(43) Pub. Date: Dec. 3, 2009

(54) Medical Punch

(76) Inventors: Martin Oberlaender, Engen (DE); Michael Sauer, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 12/474,914

(22) Filed: May 29, 2009

(30) Foreign Application Priority Data
May 31, 2008 (DE) 10 2008 026 353.2

(51) Int. Cl. A61B 17/32

(52) U.S. Cl. 606/184

(57) ABSTRACT

A medical punch having a hollow shaft, a punching tool that is mounted on the distal end of the hollow shaft and consists of a rigid cutting edge and a cutting edge that can slide with respect to the rigid cutting edge, and a handle mounted on the proximal end of the hollow shaft, where the displaceable cutting edge and the handle are operatively connected with one another by a push/pull device that is displaceably mounted in the hollow shaft and configured as a push/pull rod. To produce a medical punch that is of simple construction and can be thoroughly cleaned, it is proposed with the invention that the push/pull rod should be configured as a hollow shaft which can be inserted into the hollow shaft and extracted from the hollow shaft by the proximal end of the hollow shaft.

### CROSS-REFERENCE TO RELATED APPLICATIONS

The present application claims priority of German patent application No. 10 2008 026 353.2 filed on May 31, 2008, the content of which is incorporated herein by reference.

### FIELD OF THE INVENTION

The invention relates to a medical punch having a hollow shaft, a punching tool positioned on the distal end of the hollow shaft and consisting of a rigid cutting edge and a cutting edge that can be displaced with respect to the rigid cutting edge, and a handle positioned on the proximal end of the hollow shaft, where the displaceable cutting edge and the handle are operatively connected with one another by means of a push/pull device that is positioned so that it can slide inside the hollow shaft and configured as a push/pull rod.

### BACKGROUND OF THE INVENTION

Medical punches are cutting tools with a rigid cutting edge and a cutting edge that can move with respect to the rigid cutting edge, where the two cutting edges are not pivotable with respect to one another around a pivot point, as is the case with a scissors or the like, but rather as a rule can be moved horizontally with respect to one another.

With medical punches known in the art, the instrument shaft configured as a sliding shaft consists of a rigid shaft part and a shaft part that is displaceably mounted in a guide on the rigid shaft part, so that the displaceable shaft part can be displaced by the handle with respect to the rigid shaft part. These punches have thoroughly proven themselves in the art, yet because of the mounting of the shaft parts upon one another, very small shaft diameters are barely capable of being produced. Furthermore these punches known in the art can be disassembled for cleaning purposes only from the distal end with a certain difficulty of assembly, making handling during cleaning particularly difficult.

A generic medical punch is disclosed, for instance, in patent EP 1 055 397 A1.

It is consequently the object of the invention to configure a medical punch of the aforementioned type in such manner that it is of simple construction and also can be thoroughly cleaned.

### SUMMARY OF THE INVENTION

This object is achieved in a manner characterized in that the push/pull rod is configured as a hollow tube, which can be inserted into the hollow shaft and can be extracted from the hollow shaft by the proximal end of the hollow shaft.

This configuration of the punch as an instrument with hollow shaft, and with a push/pull rod displaceably mounted in the hollow shaft, has the advantage that the push/pull rod is removable from the hollow shaft for cleaning purposes, so that on the one hand the hollow shaft is cleanable separately, for instance by an integrated rinsing connection, and on the other hand the push/pull rod is cleanable separately.

Because of the inventive configuration of the push/pull rod as a hollow tube, which can be inserted into the hollow shaft and extracted from the hollow shaft by the proximal end of the hollow shaft, the push/pull rod can be cleaned easily and thoroughly, for example by simple rinsing.

According to the invention the push/pull rod can advantageously be coupled with the handle, in particular with the pivotable gripping part of the handle, by means of a coupling nut mounted on the handle or on the push/pull rod.

To ensure that, especially with the cutting edges of the punching tool that are configured at an angle to the shaft's longitudinal axis, the push/pull rod equipped with the displaceable cutting edge that is mounted on the proximal end is inserted in the hollow shaft always correctly positioned to the rigid cutting edge, it is proposed with the invention that mutually corresponding positioning elements should be configured on the push/pull rod and on the handle and that said elements should allow coupling of the two components that are to be connected to one another only in the correct position.

It is further proposed with the invention that the positioning elements should be configured as a pin configured on one component and as a recess configured in the other component for inserting the pin.

According to a practical embodiment of the invention it is proposed that the rigid cutting edge should be configured on the hollow shaft in the area of the distal end. The configuration of the rigid cutting edge on the hollow shaft allows the one rigid cutting edge of the punching tool to be configured in a simple manner. It is proposed with a preferred embodiment of the invention that, to configure the rigid cutting edge on the hollow shaft, in the area of the distal end of the hollow shaft a radially pointed recess should be configured, whose edge pointed toward the proximal end forms the rigid cutting edge.

It is proposed according to the invention that, to configure the displaceable cutting edge of the punching tool, the displaceable cutting edge should form the distal end of the push/pull rod mounted in the hollow shaft. Owing to the configuration of the rigid cutting edge on the hollow shaft and of the displaceable cutting edge on the distal end of the push/pull rod that is displaceably mounted in the hollow haft, the two cutting edges of the punching tool should be constructed in an especially simple manner.

It is further proposed with a first embodiment of the invention that the rigid cutting edge and the displaceable cutting edge should be configured parallel to one another and basically perpendicular to the longitudinal axis of the shaft.

According to a second embodiment of the invention it is proposed that the rigid cutting edge and the displaceable cutting edge should be configured likewise parallel to one another, but at an angle other than a right angle, preferably at 45 degrees, to the longitudinal axis of the shaft.

It is finally proposed with the invention that the handle should consist of a rigid gripping part and a gripping part that can pivot with respect to the rigid gripping part, so that the push/pull rod mounted in the hollow shaft is coupled with the pivotable gripping part. Configuring the handle with the two gripping parts allows a secure handling of the punch and a calibrated displacement of the push/pull rod coupled with the pivotable gripping part inside the hollow shaft, which in turn makes possible a calibrated, precise actuation of the punching tool.

Further characteristics and advantages of the invention can be seen from the appended illustrations, in which two embodiments of an inventive medical punch are depicted by way of example, without restricting the invention to these embodiments.

### BRIEF DESCRIPTION OF THE DRAWINGS

FIG. 1 shows a partly cut-out side view of a first embodiment of an inventive medical punch.

FIG. 2 shows an enlarged view of detail II of FIG. 1.

FIG. 3 shows a view according to FIG. 2 but depicting a second embodiment.

FIG. 4 shows a perspective view of the punch of FIG. 1, but in dismantled condition.

FIG. 5 shows a perspective view according to FIG. 4, but depicting the punch in assembled condition.

FIG. 6 shows an enlarged view of detail VI of FIG. 5.

FIG. 7 shows a side view of the detail view according to FIG. 6.

FIG. 8 shows an enlarged view of detail VIII according to FIG. 5.

FIG. 9 shows a side view of the detail view of FIG. 8.

### DETAILED DESCRIPTION OF THE INVENTION

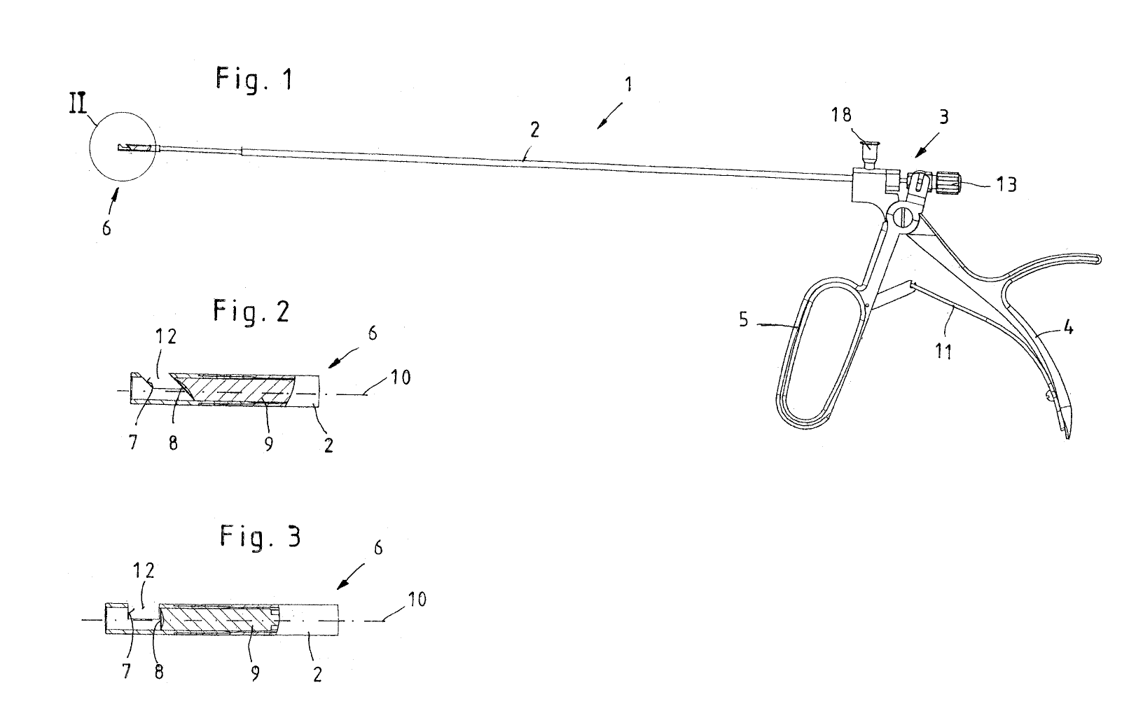

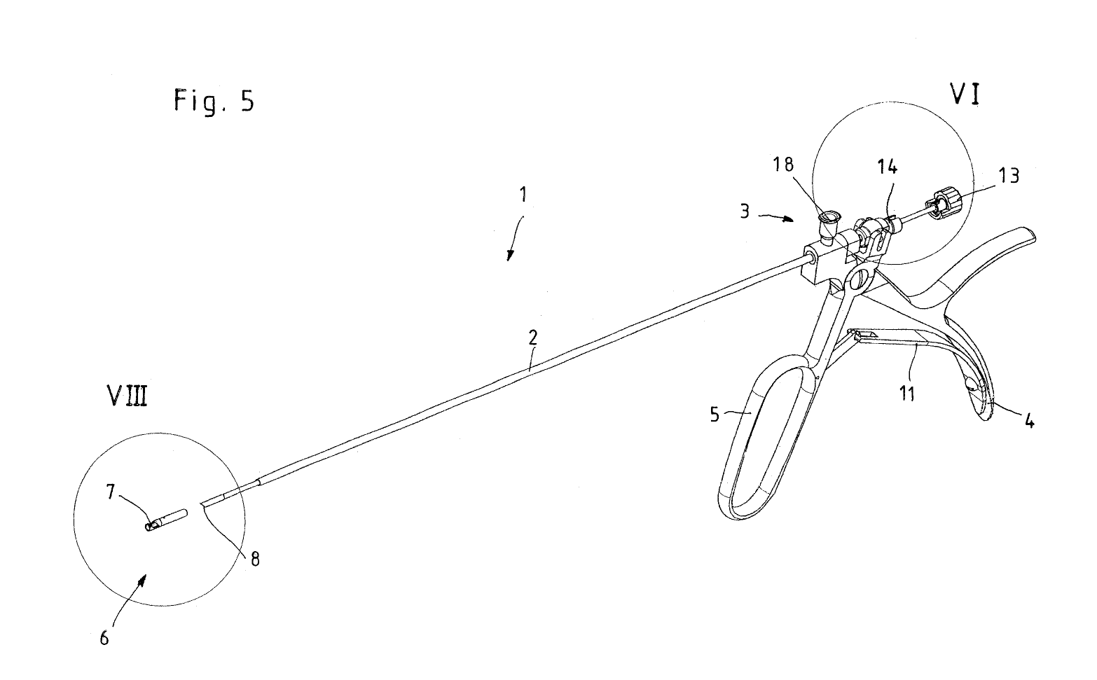

The medical punch 1 shown in its entirety in FIGS. 1, 3, 4, and 5 consists essentially of a shaft 2 configured on the hollow shaft and having on its proximal end a handle 3 which consists of a rigid gripping part 4 and a gripping part 5 that can pivot with respect to the rigid gripping part 4. On the distal end of the shaft 2 a punching tool 6 is positioned that consists of a rigid cutting edge 7 and a cutting edge 8 that can pivot with respect to the rigid cutting edge 7, where the displaceable cutting edge 8 is positioned on the distal end of a hollow push/pull rod 9 that is mounted displaceably in the hollow shaft 2.

As can be seen from FIGS. 1 and 2, the displaceable cutting edge 8 of the punching tool 6 and the pivotable gripping part 5 of the handle 3 are operatively connected with one another by means of the push/pull device configured as a push/pull rod and mounted in the hollow shaft 2 in such a way that because of the displacement of the pivotable handle grip 5 of the handle 3 the displaceable cutting edge 8 can be slid by displacing the push/pull rod 9 in the direction of the longitudinal axis 10 of the shaft 2 toward the rigid cutting edge 7 of the punching tool 6 or conversely away from the rigid cutting edge 7.

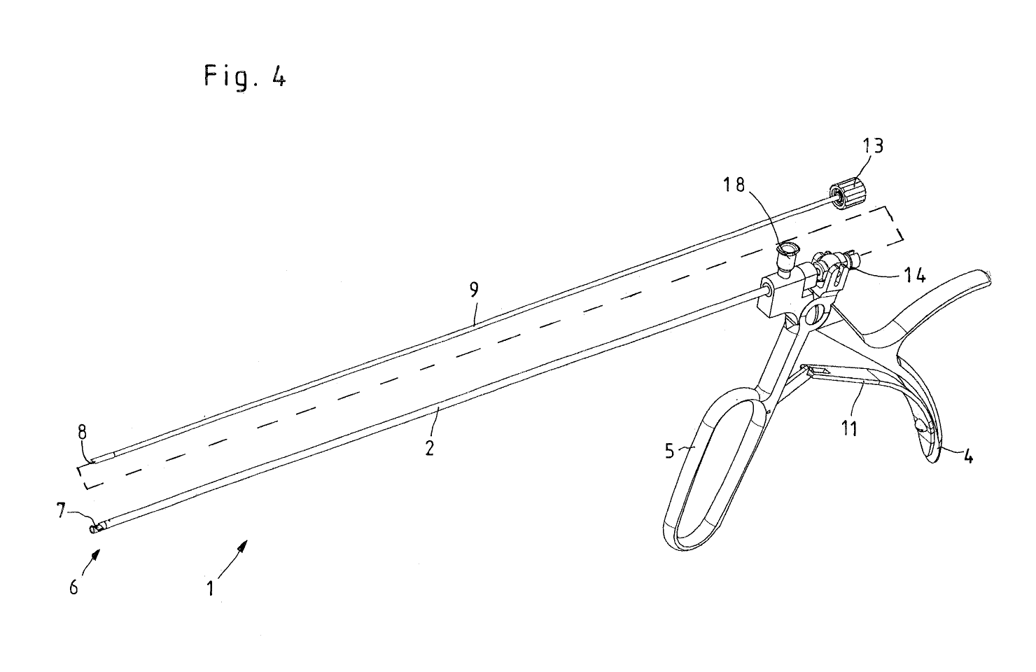

In the illustrated embodiment, pressing together the gripping parts 4 and 5 of the handle 3 causes a displacement of the push/pull rod 9 in the distal direction and thereby a sliding of the displaceable cutting edge 8 in the direction of the rigid cutting edge 7. Between the two gripping parts 4 and 5 of the handle 3 a spring element 11 is positioned, which presses the gripping parts 4 and 5 apart and thereby tenses the push/pull rod 9 in the proximal direction in which the displaceable cutting edge 8 and the rigid cutting edge 7 are at the farthest distance from one another and assume the positions with respect to one another shown in FIGS. 2 and 3.

The structure and arrangement of the cutting edges 7 and 8 of the punching tool 6 can be seen in particular from FIGS. 2 and 3, which show two embodiments for configuring the cutting edges 7 and 8. In both cases the displaceable cutting edge 8 forms the distal end of the hollow push/pull rod 9 displaceably mounted in the hollow shaft 2.

To configure the rigid cutting edge 7, in the area of the distal end of the hollow shaft 2 there is a radially pointing recess 12 configured in the hollow shaft 2 whose edge pointing in the proximal direction forms the rigid cutting edge 7.

The two illustrated embodiments for configuring the rigid and displaceable cutting edges 7 and 8 are distinguished from one another by the orientation of the cutting edges 7 and 8 to the longitudinal axis 10 of the shaft 2. The cutting edges 7 and 8 are oriented parallel to one another in both embodiments.

As can be seen from FIGS. 1 and 2, the rigid cutting edge 7 and the displaceable cutting edge 8 in the first embodiment are configured parallel to one another and at an angle other than a right angle, preferably at an angle of 45 degrees, to the longitudinal axis 10 of the shaft 2. In the second embodiment shown in FIG. 3, the rigid cutting edge 7 and the displaceable cutting edge 8 are configured basically at a right angle to the longitudinal axis 10 of the shaft 2.

The manner of assembling and dismantling of a medical punch 1 in this configuration is described hereafter, in particular with reference to FIGS. 4 through 9.

To assemble the medical punch 1, starting from the disassembled condition illustrated in FIG. 4, the hollow push/pull rod 9 is first pushed from the proximal end into the hollow shaft 2 and then is coupled with the pivotable gripping part 5 of the handle 3 in such a way that actuation of the pivotable gripping part 5 causes a longitudinal displacement of the push/pull rod 9 inside the hollow shaft 2.

In the illustrated embodiment the push/pull rod 9 is secured to the pivotable gripping part 5 of the handle 3 by means of a coupling nut 13 which is screwed onto a corresponding threaded area 14 of the handle 3. It is also possible, of course, to mount the coupling nut 13 on the handle 3 and to configure the threaded area on the proximal end of the push/pull rod 9.

In particular with the configuration of the cutting edges 7 and 8 of the punching tool 6 which is at an angle other than a right angle to the longitudinal axis 10 of the shaft 2, it is imperative that the push/pull rod 9 that bears the displaceable cutting edge 8 is inserted into the hollow shaft 2 at the correct orientation to the rigid cutting edge 7 configured on the hollow shaft 2. To ensure that the insertion of the push/pull rod 9 into the hollow shaft 2 occurs at the proper place and in the right position, mutually corresponding positioning elements 15 are configured on the push/pull rod 9 and on the handle 3 so as to prevent incorrect positioning.

In the illustrated embodiment the positioning elements 15 are configured as a pin 16 mounted on the push/pull rod 9 and a recess configured in the handle 3 for inserting the pin 16. Only when the pin 16 has been pushed completely into the recess 17 does the coupling nut 13 allow itself to be screwed onto the handle 3 on the threaded area 14 to secure the push/pull rod 9 to the handle 3. It is also possible, of course, to configure the pin 16 and the recess 17 on the other corresponding component 3 and 9.

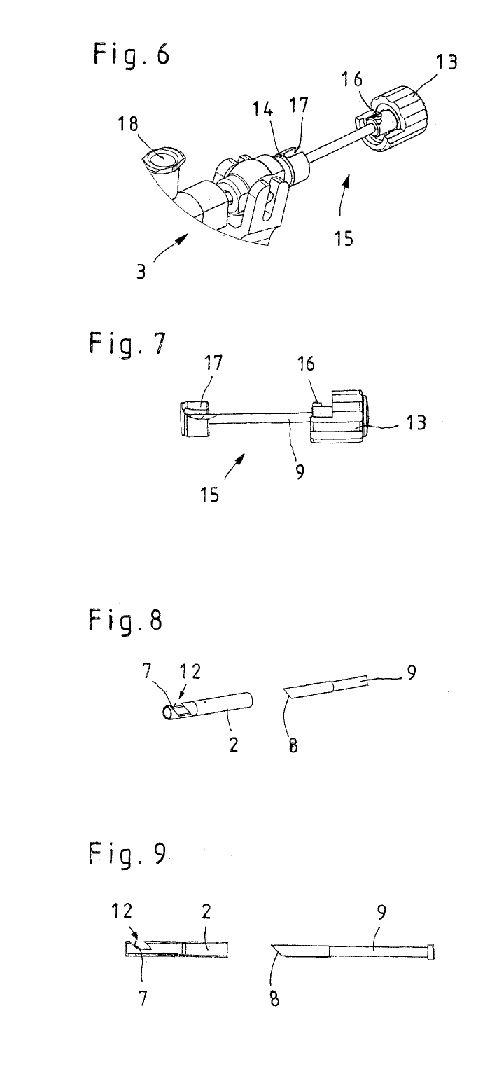

The proper alignment of the cutting edges 7 and 8 of the punching tool 6 to one another, thanks to the positioning elements 15, can be seen in particular from FIGS. 6 through 9.

After inserting the push/pull rod 9, properly aligned, into the hollow shaft 9 and after securing the push/pull rod 9 to the pivotable gripping part 5 of the handle 3 by means of the coupling nut 13, the medical punch 1 is ready for use.

The medical punch 1 is disassembled, for instance for cleaning purposes, in reverse order and is triggered by the unscrewing of the coupling nut 13 from the threaded area 14 and thus by the release of the coupling between the push/pull rod 9 and the handle 3.

Next the push/pull rod can be withdrawn from the hollow shaft 2 by the proximal end of the shaft 2. The hollow shaft 2 is cleaned, for instance, by a rinsing connection 18 mounted on the handle 3. The push/pull rod 9 extracted from the hollow shaft 2 can likewise, be cleaned easily and thoroughly as a separate component.

A medical punch 1 configured in this manner is characterized in that on the one hand it has a simple structure, which allows the production of very small shaft diameters, and on the other hand can easily be washed.

1. A medical punch having a hollow shaft, a punching tool positioned on the distal end of the hollow shaft and consisting of a rigid cutting edge and a cutting edge that can slide with respect to the rigid cutting edge, and a handle positioned on the proximal end of the hollow shaft, where the displaceable cutting edge and the handle are operatively connected to one another by means of a push/pull device configured as a push/pull rod and mounted displaceably in the hollow shaft, wherein the push/pull rod is configured as a hollow tube which can be inserted into the hollow shaft and withdrawn from the hollow shaft by the proximal end of the hollow shaft.

2. The medical punch according to claim 1, wherein the push/pull rod can be secured to the handle by a coupling nut.

3. The medical punch according to claim 1, wherein mutually corresponding positioning elements (are configured on the push/pull rod and on the handle.

4. The medical punch according to claim 3, wherein the positioning elements are configured as a pin configured on a component and a recess configured in the other component for inserting the pin.

5. The medical punch according to claim 1, wherein the rigid cutting edge is configured on the hollow shaft in the area of the distal end.

6. The medical punch according to claim 5, wherein in the area of the distal end of the hollow shaft a radially pointing recess is configured, whose edge pointing in the proximal direction forms the rigid cutting edge.

7. The medical punch according to claim 1, wherein the displaceable cutting edge forms the distal end of the push/pull rod mounted in the hollow shaft.

8. The medical punch according to claim 1, wherein the rigid cutting edge hand the displaceable cutting edge are configured parallel to one another and essentially at a right angle to the longitudinal axis of the shaft.

9. The medical punch according to claim 1, wherein the rigid cutting edge and the displaceable cutting edge are configured parallel to one another and at an angle other than a right angle, preferably at 45 degrees, to the longitudinal axis of the shaft.

10. The medical punch according to claim 1, wherein the handle consists of a rigid gripping part and a gripping part that can pivot with respect to the rigid gripping part, where the push/pull rod mounted in the hollow shaft is coupled with the pivotable gripping part.
